(19) United States
(12) Patent Application Publication
(10) Pub. No.: US 2021/0137372 A1
(43) Pub. Date: May 13, 2021

(54) IMAGE PICKUP APPARATUS FOR ENDOSCOPE AND ENDOSCOPE

(71) Applicant: OLYMPUS CORPORATION, Tokyo (JP)

(72) Inventor: Noriyuki FUJIMORI, Suwa-shi (JP)

(73) Assignee: OLYMPUS CORPORATION, Tokyo (JP)

(21) Appl. No.: 17/123,271

(22) Filed: Dec. 16, 2020

Related U.S. Application Data
(63) Continuation of application No. PCT/JP2018/025671, filed on Jul. 6, 2018.

(51) Int. Cl. A61B 1/05; H01L 27/146; H04N 5/225; A61B 1/00

(52) U.S. Cl. CPC A61B 1/05; H01L 27/14627; H04N 2005/2255; A61B 1/00163; H04N 5/2254

(57) ABSTRACT

An image pickup apparatus for endoscope includes an optical member that is a hybrid lens element in which a plurality of optical elements are bonded to each other, at least any one of the plurality of optical elements including a resin lens disposed on a principal surface of a parallel flat glass plate, and an image pickup member that receives an object image brought into focus by the optical member. A surface of the resin lens and the principal surface around the resin lens are covered with a transparent inorganic film such that a boundary between the surface of the resin lens and the principal surface around the resin lens is also covered with the inorganic film.

### CROSS REFERENCE TO RELATED APPLICATION

This application is a continuation application of PCT/JP2018/025671 filed on Jul. 6, 2018, the entire contents of which are incorporated herein by this reference.

### BACKGROUND OF THE INVENTION

### 1. Field of the Invention

The present invention relates to an image pickup apparatus for endoscope including an optical member including a resin lens and also relates to an endoscope including the image pickup apparatus for endoscope including an optical member including a resin lens.

### 2. Description of the Related Art

It is important for an image pickup apparatus for endoscope to reduce the size of the apparatus for low invasiveness.

As a method for efficiently manufacturing a compact image pickup apparatus, there is a wafer-level production method of cutting a bonded wafer formed of a plurality of bonded element wafers each including a plurality of optical elements.

Japanese Patent Application Laid-Open Publication No. 201-18993 discloses an image pickup module formed of a wafer-level laminate. The image pickup module is produced by bonding an optical wafer including a plurality of optical elements to an image pickup wafer including a plurality of image pickup devices and then cutting the bonded wafer into individual pieces.

Japanese Patent Application Laid-Open Publication No. 2015-38538 discloses what is called a hybrid lens element in which lenses made of resin are disposed on a parallel flat glass plate.

A compact image pickup apparatus can be efficiently manufactured by the wafer-level method to produce an optical member including a plurality of hybrid lens elements.

### SUMMARY OF THE INVENTION

An image pickup apparatus for endoscope according to an embodiment of the present invention includes an optical member that is a hybrid lens element in which a plurality of optical elements are bonded to each other, at least any one of the plurality of optical elements including a resin lens disposed on a principal surface of a parallel flat glass plate and an image pickup member that receives an object image brought into focus by the optical member. A surface of the resin lens and the principal surface around the resin lens are covered with a transparent inorganic film such that a boundary between the surface of the resin lens and the principal surface around the resin lens is also covered with the transparent inorganic film.

An endoscope according to another embodiment includes an image pickup apparatus for endoscope, and the image pickup apparatus for endoscope includes an optical member that is a hybrid lens element in which a plurality of optical elements are bonded to each other, at least any one of the plurality of optical elements including a resin lens disposed on a principal surface of a parallel flat glass plate and an image pickup member that receives an object image brought into focus by the optical member. A surface of the resin lens and the principal surface around the resin lens are covered with a transparent inorganic film such that a boundary between the surface of the resin lens and the principal surface around the resin lens is also covered with the transparent inorganic film.

### BRIEF DESCRIPTION OF THE DRAWINGS

FIG. 1 is a perspective view of an endoscope system including an endoscope according to an embodiment;

FIG. 2 is a perspective view of an image pickup apparatus for endoscope according to the embodiment;

FIG. 3 is a cross-sectional view of the image pickup apparatus for endoscope according to the embodiment taken along a line in FIG. 2;

FIG. 4 is an exploded view of an optical element in the image pickup apparatus for endoscope according to the embodiment;

FIG. 5 is a flowchart for describing a method for manufacturing the image pickup apparatus for endoscope according to the embodiment;

FIG. 6 is a cross-sectional view of an optical element wafer for describing the method for manufacturing the image pickup apparatus for endoscope according to the embodiment;

FIG. 7 is a cross-sectional view of the optical element wafer for describing the method for manufacturing the image pickup apparatus for endoscope according to the embodiment;

FIG. 8 is a cross-sectional view of the optical element wafer for describing the method for manufacturing the image pickup apparatus for endoscope according to the embodiment;

FIG. 9 is a cross-sectional view of the optical element wafer for describing the method for manufacturing the image pickup apparatus for endoscope according to the embodiment;

FIG. 10 is a cross-sectional exploded view of a bonded wafer for describing the method for manufacturing the image pickup apparatus for endoscope according to the embodiment;

FIG. 11 is a cross-sectional view of the bonded wafer for describing the method for manufacturing the image pickup apparatus for endoscope according to the embodiment;

FIG. 12 is a cross-sectional view of the bonded wafer for describing the method for manufacturing the image pickup apparatus for endoscope according to the embodiment;

FIG. 13 is a cross-sectional view of an image pickup apparatus for endoscope according to Modification 1 of the embodiment;

FIG. 14 is a cross-sectional view of an image pickup apparatus for endoscope according to Modification 2 of the embodiment;

FIG. 15 is a plan view of a concave lens optical element of an image pickup apparatus for endoscope according to Modification 3 of the embodiment; and

FIG. 16 is a plan view of a concave lens optical element of an image pickup apparatus for endoscope according to Modification 4 of the embodiment.

### DETAILED DESCRIPTION OF THE PREFERRED EMBODIMENT

### Embodiment

An endoscope system 8 including an endoscope 9 according to an embodiment includes the endoscope 9, a processor 80, a light source apparatus 81, and a monitor 82, as shown in FIG. 1. In the operation of the endoscope 9, an insertion section 90 is inserted into a body cavity of a subject, an in-body image of the subject is captured, and an image pickup signal is outputted.

A proximal end portion of the insertion section 90 of the endoscope 9 is provided with an operation section 91 provided with various buttons for operating the endoscope 9. The insertion section 90 is formed of a rigid distal end section 90A, where the image pickup apparatus 1 for endoscope (hereinafter referred to as “image pickup apparatus 1”) is disposed, a bending section 90B, which is bendable and is provided continuously to the proximal end portion of the rigid distal end section 90A, and a flexible section 90C, which is provided continuously to a proximal end portion of the bending section 90B. The bending section 90B bends when operated by the operation section 91.

A universal cord 92, which extends from the operation section 91, is connected to the processor 80 and the light source apparatus 81 via connectors 93. A signal cable 94, which transmits an electric signal outputted by the image pickup apparatus 1, is inserted through the insertion section 90, the operation section 91, and the universal cord 92.

The processor 80 controls the entire endoscope system 8, performs signal processing on the image pickup signal emitted by the image pickup apparatus 1, and outputs the processed signal as an image signal. The monitor 82 displays the image signal outputted by the processor 80.

The light source apparatus 81 includes, for example, a white LED. Illumination light emitted by the light source apparatus 81 is guided to the rigid distal end section 90A via a light guide (not shown) inserted through the universal cord 92 and the insertion section 90 to illuminate an object.

The image pickup apparatus 1 of the endoscope 9 is compact and excels in moisture resistance, as will be described later, and the endoscope 9 therefore provides low invasiveness and excels in moisture resistance.

While the endoscope 9 is a flexible endoscope for medical use, an endoscope according to another embodiment may be a rigid endoscope or an endoscope for industrial use.

### <Configuration of Image Pickup Apparatus for Endoscope>

The image pickup apparatus 1 for endoscope according to the embodiment includes an optical member 10 and an image pickup member 50, as shown in FIGS. 2 and 3.

Note in the following description that the figures based on each embodiment are diagrammatic and the relationship between the thickness and the width of each portion, the thickness ratio among portions, and other factors differ from values in the actual endoscope, and that the dimensions and the proportions of each portion vary among the figures in some cases. Further, some of the components are not shown and no reference characters are provided in some cases. The direction toward an object is referred to as an upward direction.

The optical member 10 is a wafer-level laminate in which a plurality of optical elements 20, 29, 30, 39, 40, and 49 are bonded to each other. In other words, the optical member 10 is produced by cutting a bonded wafer 10W (see FIG. 10) formed of a plurality of optical element wafers boned to each other, so that a side surface 10SS has cut traces, and outer dimensions of the cut optical members 10 in the direction perpendicular to an optical axis are the same or substantially the same (for example, within ±5% of average outer dimension).

The optical elements 20, 30, and 40 are hybrid lens elements in which resin lenses 22, 32, and 42 are disposed on parallel flat glass plates (hereinafter referred to as “glass plate”) 21, 31, and 41, respectively. A transparent resin for lens that forms the resin lenses 22, 32, and 42 is, for example, an acrylic resin.

The image pickup member 50, which includes an image pickup device 51 and a cover glass 52 with the cover glass 52 bonded to the optical member 10, receives an object image brought into focus by the optical member 10.

The gaps between the plurality of optical elements 20, 30, and 40 and the image pickup member 50 are defined by the optical elements 29, 39, and 49, which are each a spacer. The optical elements 29, 39, and 49 are made, for example, of silicon, and an area of each of the optical elements that forms an optical path is a space. The optical elements 29, 39, and 49 may each instead be made of glass having a predetermined shape.

The optical element 20 is a hybrid lens element formed of a glass plate 21 having a first principal surface 21SA and a second principal surface 21SB, and an aspheric concave lens 22 made of resin and disposed on the second principal surface 21SB. The first principal surface 21SA is a front surface 10SA of the optical member 10. The optical element 30 is a hybrid lens element formed of a glass plate 31 having a third principal surface 31SA and a fourth principal surface 31SB, and an aspheric convex lens 32 made of resin and disposed on the third principal surface 31SA. The optical element 40 is a hybrid lens element formed of a glass plate 41 having a fifth principal surface 41SA and a sixth principal surface 41SB, and an aspheric convex lens 42 made of resin and disposed on the sixth principal surface 41SB.

The configuration of the optical member 10, that is, the types, the number, and the lamination order of the optical elements can be changed in a various manners in accordance with the specifications of the optical member 10. For example, a patterned light blocking film having an aperture function may be disposed on the principal surface of any of the optical elements.

In the image pickup apparatus 1, the entire surfaces of all the resin lenses 22, 32, and 42 and the principal surfaces 21SB, 31SA, and 41SB around all the resin lenses 22, 32, and 42 are covered with transparent inorganic films 23, 33, and 43, which are made of a transparent inorganic material and extend over the boundaries between the surfaces of the resin lenses 22, 32, and 42 and the principal surfaces 21SB (21SB2), 31SA, and 41SB around the resin lenses 22, 32, and 42.

No resin lens is disposed at the first principal surface 20SA of the optical element 20, which is a first optical element disposed in a frontmost portion closest to the object among the plurality of optical elements 20, 30, and 40, that is, the front surface 10SA of the optical member 10.

When the image pickup apparatus 1 is accommodated in the rigid distal end section 90A of the endoscope 9, the side surface 10SS of the optical member 10 is not exposed to an external environment. The front surface 10SA of the optical member 10 (first principal surface 21SA of optical element 20) is, however, exposed to the external environment. The reliability of a resin lens, even when covered with a transparent inorganic film, is not sufficient as compared with the reliability of a glass lens. Further, an exposed resin lens can be broken.

The image pickup apparatus 1 is highly reliable because no resin lens is disposed on the front surface (first principal surface 20SA) of the optical element 20.

The hybrid lens element will be described below with reference to the optical element 20 shown in FIG. 4 as an example. The resin lens 22, which is a concave lens, is so configured that the surface formed of an optical path surface X22, which is a concave surface that forms the optical path, an outer circumferential surface Y22, which surrounds the optical path surface X22, and an outer side surface Z22 is covered with the transparent inorganic film 23. A bonding surface A22, which faces the optical path surface X22 and the outer circumferential surface Y22, is molded integrally with the second principal surface 21SB1 (21SB) of the glass plate 21.

To completely cover the outer side surface Z22 of the resin lens 22 and to prevent detachment of the resin lens 22 from the glass plate 21, the second principal surface 21SB2 (21SB) around the resin lens 22 is also covered with the transparent inorganic film 23. The resin lens 22 has high bonding reliability because the outer side surface Z22, which is an edge portion of the outer circumferential surface Y22, which becomes a start point of the detachment, is covered with the transparent inorganic film 23, which is so disposed as to extend over the boundary between the outer side surface Z22 and the second principal surface 21SB (21SB2).

The optical element 30, which is a convex lens, is so configured, as the optical element 20 is, that the entire surface of the resin lens 32 and the third principal surface 31SA around the resin lens 32 are covered with a transparent inorganic film 33, although not shown in the form of an exploded view. The optical element 40, which is a convex lens, is also so configured that the entire surface of the resin lens 42 and the sixth principal surface 41SB around the resin lens 42 are covered with the transparent inorganic film 43.

In other words, the resin lenses 22, 32, and 42 each have no surface exposed to the external environment because the entire surfaces of the resin lenses 22, 32, and 42 are covered with the transparent inorganic films 23, 33, and 43 and the glass plates 21, 31, and 41.

Further, all the bonded portions of the plurality of optical elements 20, 29, 30, 39, 40, and 49 are directly bonded to each other using no adhesive. The direct bonding is a technology for bonding two substrates, for example, only by carrying out an activation process of removing contaminants on surfaces to be bonded to each other or forming dangling bond by a vacuum plasma process, and pressure joining the activated surfaces with each other. After the bonding at room temperature, a heat treatment can further be performed to increase the bonding strength. To perform the direct bonding, a metal film or an inorganic film made, for example, of silicon oxide may be deposited on each of the surfaces to be bonded to each other.

The entire surfaces of the resin lenses 22, 32, and 42 are completely covered with the transparent inorganic films 23, 33, and 43, which excel in moisture permeability as compared with a transparent resin for lens, and the glass plates 21, 31, and 41. Further, the plurality of bonded portions of the optical member 10 are directly bonded to each other and contain no organic material having high moisture permeability.

The image pickup apparatus 1, which includes the optical member 10 formed of the wafer-level laminate, is compact. Further, the resin lenses 22, 32, and 42, which are covered with the transparent inorganic film 43 having low moisture permeability, excel in moisture resistance. The endoscope 9 including the image pickup apparatus 1 provides low invasiveness and excels in moisture resistance.

The bonded portion where the optical member 10 is bonded to the image pickup member 50 and the bonded portion where the image pickup device 51 is bonded to the cover glass 52 preferably contain no organic material having high moisture permeability, as will be described later.

### <Method for Manufacturing Image Pickup Apparatus>

A method for manufacturing the image pickup apparatus 1 for endoscope will next be described with reference to the flowchart shown in FIG. 5.

### <Step S10> Optical Element Wafer Producing Step

For example, a glass wafer 21W, which is a parallel flat glass plate, is prepared, as shown in FIG. 6. The thickness of the glass wafer 21W is determined in accordance with the specifications of the image pickup apparatus 1. The glass wafer 21W is cut into the glass plates 21 of the optical elements 20.

A plurality of resin lenses 22 are then disposed in predetermined positions on the second principal surfaces 20SB of the glass wafer 21W. For example, a transparent resin for lens is applied onto the glass wafer 21W, and a die having a predetermined shape (not shown) is pressed against each of the second principal surfaces 21SB. In this state, ultraviolet (UV) light is radiated to the transparent resin for lens from the direction facing the first principal surfaces 21SA to harden the transparent resin for lens. The molded resin lenses 22 are thus disposed on the second principal surfaces 21SB.

The resin lenses 22 are each a concave lens and each have the optical path surface X22, the outer circumferential surface Y22, which surrounds the optical path surface X22, and the outer side surface Z22. In the image pickup apparatus 1, the plurality of resin lenses 22 each have the outer circumferential surface Y22 because the outer circumferential surfaces Y22 of the resin lenses 22 are separate from each other with a gap between the resin lenses 22.

A silicon oxide film (SiO₂) film is deposited as a transparent inorganic film 23W on the entire second principal surface 21SB of the glass wafer 21W on which the plurality of resin lenses 22 are disposed, as shown in FIG. 7. In other words, the transparent inorganic film 23W covers the entire surfaces of the plurality of resin lenses 22 and the second principal surfaces 20SB of the glass wafer 21W around the plurality of resin lenses 22.

The transparent inorganic film preferably has moisture permeability, which is used in water vapor permeability test specified in JIS Z 0208, smaller than or equal to 5 g/(m²×day), particularly preferably smaller than or equal to 1 g/(m²×day) even in a thinnest film thickness area. Further, the transparent inorganic film preferably has transmittance higher than or equal to 90% at a wavelength of the light to be brought into focus. The film thickness on the optical path surface X22 is preferably greater than or equal to 0.1 μm but smaller than or equal to 10 μm, and variation in film thickness preferably falls within ±10% of the film thickness.

When the film thickness is greater than or equal to the range described above, the moisture permeability improvement effect is secured. When the film thickness and the variation in film thickness are smaller than or equal to the ranges described above, the surface of the resin lens 22, in particular, the shape of the optical path surface X22 does not change, and the optical characteristics of the resin lens 22 therefore do not deteriorate.

The transparent inorganic film 23W may be a multilayer film formed of a plurality of transparent inorganic layers as long as the multilayer film has the characteristics described above.

For example, the transparent inorganic film 23W is a monolayer film made of at least one of aluminum oxide (A1₂O₃), silicon nitride (SiN), magnesium oxide (MgO), niobium oxide (Nb₂O₅), silicon oxide (SiO₂), tantalum oxide (Ta₂O₅), titanium oxide (TiO₂), zirconium oxide (ZrO₂), magnesium fluoride (MgF₂), antimony sulfide (Sb₂S₃), and zinc sulfide (ZnS) or a multilayer film made of a plurality of the materials.

The transparent inorganic film 23W is preferably deposited at a temperature lower than or equal to a softening point of the resin lens 22, for example, ranging from 100 to 200° C. to prevent deterioration or deformation of the resin lens 22. The transparent inorganic film 23W is disposed, for example, by using CVD, sputtering, evaporation, or dehydration synthesis using, for example, alkoxysilane (water glass).

As described above, an optical element wafer 20W is produced by disposing a plurality of resin lenses 22 on the glass wafer 21W and depositing the transparent inorganic film 23W that covers the resin lenses 22. Similarly, an optical element wafer 30W is produced by disposing a plurality of resin lenses 32 on a glass wafer 31W and depositing a transparent inorganic film 33W that covers the resin lenses 32 (see FIG. 10). Further, an optical element wafer 40W is produced by disposing a plurality of resin lenses 42 on a glass wafer 41W and depositing a transparent inorganic film 43W that covers the resin lenses 42 (see FIG. 10).

The optical element wafers 20W, 30W, and 40W may instead so configured that the resin lenses 22, 32, and 42 are made of different resins, and that different transparent inorganic films 23W, 33W, and 43W are deposited.

On the other hand, in the step of producing an optical element wafer 29W, which serves as a plurality of spacers 29, an etching mask 28 for forming spaces that serve as the optical paths is disposed, for example, on a principal surface of a silicon wafer 29W, as shown in FIG. 8. A support substrate 27 is glued to a principal surface (rear surface) that faces the principal surface on which the etching mask 28 is disposed.

The optical element wafer 29W, which is a spacer wafer having a plurality of through holes H29, which each serve as the space that serves as the optical path, is produced by using dry etching based, for example, on ICP-RIE or wet etching using an alkali solution made, for example, of KOH or TMAH. The through holes H29 may instead be formed by physical processing, such as laser processing. An inner diameter of the through holes H29 in the direction perpendicular to the optical axis is so set as to be slightly greater than an outer diameter of the outer circumferential surface Y22 of the resin lens 22.

Optical element wafers 39W and 49W, which serve as a plurality of spacers 39 and 49, are produced by the same method used to produce the optical element wafer 29W (see FIG. 10).

The material of the optical element wafers 29W, 39W, and 49W is not limited to silicon and may be ceramic, glass, metal, or resin, as will be described later. To perform the direct bonding, a silicon oxide film or any other film is deposited on a principal surface of a wafer made of metal or resin.

Further, for example, the transparent inorganic film 23W may be deposited after the silicon wafer 29W, which is a spacer wafer, is bonded to the glass wafer 21W on which a plurality of resin lenses 22 have been disposed.

### <Step S20> Optical Element Wafer Bonding Step

The optical element wafers 20W, 29W, 30W, 39W, 40W, and 49W are bonded to each other to produce the bonded wafer 10W, as shown in FIG. 10.

For example, the optical element wafers 20W, 29W, 30W, 39W, 40W, and 49W, each having a plasma-processed surface to be bonded, are bonded to each other in a pressure joining process at room temperature, and are then subjected to a heat treatment for one hour at 120° C. Note that all the optical element wafers are not necessarily bonded to each other at the same time.

### <Step S30> Image Pickup Device Gluing Step

A plurality of image pickup members 50 are glued to the bonded wafer 10W via a bonding layer (not shown), as shown in FIG. 11.

In a method for producing the image pickup members 50, light receiving sections 53, such as CMOS light receiving elements, are disposed on a light receiving surface 50SA on a semiconductor wafer by a known semiconductor manufacturing method. An image pickup device wafer (not shown) in which the light receiving sections 53 are connected to external connection electrodes 54 on a rear surface 50SB is then produced by producing through wiring (not shown). After a glass wafer is glued to the light receiving surface 50SA of the image pickup device wafer via a bonding layer (not shown), the resultant structure is cut, whereby the image pickup member 50 including the image pickup device 51 and the cover glass 52 is produced.

The bonded portion where the optical member 10 is bonded to the image pickup member 50 and the bonded portion where the image pickup device 51 is bonded to the cover glass 52 are also directly bonded to each other or bonded to each other by an inorganic adhesive containing no organic material. In other words, in the image pickup apparatus according to the present embodiment, none of the plurality of bonded portions contains an organic material.

The inorganic adhesive is selected from materials having the same moisture permeability of the transparent inorganic film. For example, the inorganic adhesive is formed of a silicon oxide layer formed by dehydration synthesis (sol-gel method) using, for example, water glass (silane alkoxide), an adhesive containing an inorganic binder, or low-molting-point glass or solder having a melting point ranging from 100 to 200° C.

The inorganic binder can, for example, be alkali metal silicate, such as sodium silicate, potassium silicate, and lithium silicate; phosphate, such as aluminum phosphate, magnesium phosphate, and calcium phosphate; or silica sol.

The inorganic adhesive may further contain as a hardening assistant, an oxide, such as zinc oxide, magnesium oxide, and calcium oxide; a hydroxide, such as zinc hydroxide, magnesium hydroxide, and calcium hydroxide; or a borate, such as calcium borate, barium borate, and magnesium borate.

The inorganic adhesive may further contain inorganic powder for preventing fracture or any other undesirable phenomenon due to thermal contraction. Examples of the inorganic powder may include ceramics such as zirconia, silica, alumina, magnesia, aluminum nitride, or yttrium oxide. The inorganic powder may be any one of the inorganic powder types, or a mixture of two or more of the inorganic powder types may be used. The inorganic powder is preferably an inorganic adhesive containing alumina and silica. The average grain diameter of the inorganic powder preferably ranges from 0.1 to 5 μm.

To bond at least any one of the plurality of bonded portions to another, the inorganic adhesive may be replaced with an adhesive layer made of organic resin having low moisture permeability, for example, moisture permeability of 30 g/(m²×day). Even when water enters the optical member via the adhesive layer, the resin lens itself is not affected by the water because the surface of the resin lens is covered with the transparent inorganic film.

All the plurality of bonded portions may or may not contain an organic material, or may be formed of a bonded portion containing no organic material and a bonded portion containing an organic material.

### <Step S40> Cutting Step

The bonded wafer 10W to which the plurality of image pickup members 50 are glued is cut to produce the image pickup apparatuses 1 each including the optical member 10 and the image pickup member 50, as shown in FIG. 12. Cutting traces, which are minute irregularities, are present at the side surface MSS of each of the optical members 10 individualized, for example, with a dicing saw. In other words, the side surface 1055 of each of the optical member 10, which are each a wafer-level structure, has cutting trances. The plurality of optical elements, which form the optical member 10, have the same or substantially the same outer shape and have the same or substantially the same outer dimensions (for example, within ±5% of average outer dimensions).

The cutting step may, for example, be a cutting step by laser dicing or individualizing step of forming cutting grooves by sand blasting or etching.

A plurality of concave lenses disposed on a glass wafer each have the function as the optical element as long as the concave lens has the optical path surface X22 even when the outer circumferential surface Y22 is a continuous surface. Even when the plurality of concave lenses with the outer circumferential surfaces Y22 being a continuous surface are covered with a transparent inorganic film, the resin that forms the individualized (cut) concave lenses is exposed on the side surfaces of the concave lenses.

In contrast, in the image pickup apparatus 1, the outer circumferential surfaces Y22 of the plurality of resin lenses 22 disposed on the glass wafer 21 are separate from each other via the gap, so that the resin lenses 22 each have the outer circumferential surface Y22. The individualized (cut) concave lenses each therefore have a side surface on which only the transparent inorganic film is exposed to the external environment.

The plurality of optical elements 20 and other components all have a rectangular parallelepiped shape and the same cross-sectional shape and size in the direction perpendicular to the optical axis, so that the optical member 10 is a quadrangular column. In the image pickup apparatus 1, the cross section of the image pickup member 50 in the direction perpendicular to the optical axis has substantially the same shape and size as the shape and size of the cross section of the optical member 10 in the direction perpendicular to the optical axis.

In other words, to reduce the size of the image pickup apparatus 1, it is preferable that the size of the cross section of the image pickup member 50 in the direction perpendicular to the optical axis is smaller than or equal to the size of the cross section of the optical member 10 in the direction perpendicular to the optical axis, and that the image pickup member 50 is accommodated in a space formed by extending the front surface 10SA of the optical member 10 in the optical axis direction.

The image pickup apparatus 1 may instead be produced by gluing the image pickup device wafer to the bonded wafer 10W and then cutting the glued wafers. In this case, the cross section of the image pickup member 50 in the direction perpendicular to the optical axis has the same shape and the same size as the shape and the size of the cross section of the optical member 10 in the direction perpendicular to the optical axis.

The manufacturing method described above allows the image pickup apparatus 1, which is compact and excels in moisture resistance, to be readily produced in large quantities. The image pickup apparatus 1 is disposed in the rigid distal end section 90A of the endoscope 9.

### <Modifications>

A description will next be made of image pickup apparatuses 1A to 1D according to modifications of the embodiment and endoscopes 9A to 9D according to modifications including the image pickup apparatuses 1A to 1D. The image pickup apparatuses 1A to 1D according to the modifications or the endoscopes 9A to 9D according to the modifications are similar to and have the same functions as the image pickup apparatus 1 or the endoscope 9, and a component having the same function therefore has the same reference character and will not be described.

### <Modification 1>

The image pickup apparatus 1A shown in FIG. 13 includes an optical member 10A including a plurality of optical elements 20A, 29, 30A, 39, 40A, and 49, and an image pickup member 50A.

The optical element 20A includes a resin lens 22A, which is a concave lens. The outer side surface Z22 around the resin lens 22A inclines with respect to an optical axis 0. The optical element 29, which is a spacer, is bonded to the outer circumferential surface Y22 of the optical element 20A. Part of the transparent inorganic film 23, which covers the outer side surface Z22 of the optical element 20A, is therefore exposed to the external environment on the side surface 10SS of the optical member 10A.

The optical element 30A is so configured that a resin lens 32A, which is a convex lens, is disposed on the third principal surface 31SA of the glass plate 31, and a resin lens 32B, which is a convex lens, is disposed on the fourth principal surface 31SB of the glass plate 3. In other words, the resin lenses 32A and 32B are disposed on the opposite principal surfaces of the glass plate 31.

The optical element 40A is an infrared removing filter element having a function of blocking infrared light and is provided with no resin lens. In other words, in the optical member 10A, only at least any one of the optical elements needs to be a hybrid lens element.

The image pickup member 50A is the image pickup device 51 and includes no cover glass. The image pickup member 50A of the image pickup apparatus 1A has a cross section that extends in the direction perpendicular to the optical axis and is smaller than the cross section of the optical member 10A.

The optical elements 20A, 29, 30A, 39, 40A, and 49 and the image pickup member 50A are glued to each other by a gluing layer made of silicon oxide.

In the image pickup apparatus 1A, the surfaces of the resin lenses 22A, 32A, and 32B and the principal surfaces 21SB, 31SA, and 31SB of the glass plates 31 and 32 around the resin lenses are covered with transparent inorganic films 23, 33A, and 33B. A plurality of bonded portions where the plurality of optical elements 20A, 29, 30A, 39, 40A, and 49 are bonded to each other by using the inorganic adhesives 28, 37, 38, 47, and 48 having been already described. The inorganic adhesive 28 preferably further covers the transparent inorganic film 23, which covers the outer side surface Z22 of the optical element 20A.

The inorganic adhesives 28, 37, 38, 47, and 48 are selected from materials having the same moisture permeability of the transparent inorganic films, whereby the image pickup apparatus 1A provides the same effects provided by the image pickup apparatus 1.

At least any one of the plurality of bonded portions may be directly bonded to another without use of any adhesive, and a bonded portion that is not directly bonded to another may be bonded with an inorganic adhesive.

### <Modification 2>

In an image pickup apparatus 1B according to Modification 2, an optical element 20B has the functions of a concave lens and a spacer, as shown in FIG. 14. The optical element 20B made of resin is covered with the transparent inorganic film 23.

The image pickup apparatus 1B requires no optical element 29, which is a spacer, as compared with the image pickup apparatus 1A, whereby the image pickup apparatus 1B has a simplified structure and is therefore readily manufactured and still provides the same effects provided by the image pickup apparatus 1A. In other words, the spacer may be a resin lens element covered with a transparent inorganic film. Still instead, the spacer may be a resin element covered with a transparent inorganic film.

### <Modifications 3 and 4>

In an image pickup apparatus 1C according to Modification 3, a resin lens 22C, which is a concave lens disposed on the second principal surface 21SB of the glass plate 21 of an optical element 20C, is so shaped that the outer side surface Z22 has a substantially rectangular shape that extends in the direction perpendicular to the optical axis and has curved corners, as shown in FIG. 15.

In an image pickup apparatus 1D according to Modification 4, a resin lens 22D, which is a concave lens disposed on the second principal surface 21SB of the glass plate 21 of an optical element 20D, is so shaped that the outer side surface Z22 has a circular shape in the direction perpendicular to the optical axis, as shown in FIG. 16.

In the image pickup apparatuses 1C and 1D, the resin lenses 22C and 22D are unlikely to be detached from the glass plates 21 and 21D as compared with the image pickup apparatus 1 including the resin lens 22, the outer side surface Z22 around which has a rectangular parallelepiped shape in the direction perpendicular to the optical axis. The image pickup apparatuses 1C and 1D therefore have higher reliability than the image pickup apparatus 1.

The glass plate 21D of the optical element 20D of the image pickup apparatus 1D has corners chamfered in the optical axis direction and has an octagonal cross-sectional shape in the direction perpendicular to the optical axis. In the image pickup apparatus 1D, the other optical elements of the optical member each have corners parallel to the optical axis chamfered as in the optical element 20D. In other words, the optical member of the image pickup apparatus 1D is not a quadrangular column but is an octagonal column.

The image pickup apparatus 1D is more compact than the image pickup apparatus 1 in such a way that the size in the direction perpendicular to the optical axis is smaller than the size of the image pickup apparatus 1.

Needless to say, the endoscopes 9A to 9D including the image pickup apparatuses 1A to 1D provide not only the effects provided by the endoscope 9 but the effects provided by the image pickup apparatuses 1A to 1D.

The present invention is not limited to the embodiments and the like described above, and various changes, combinations, and applications are possible without departing from the spirit of the present invention.

What is claimed is:

1. An image pickup apparatus for endoscope, comprising:
an optical member that is a hybrid lens element in which a plurality of optical elements are bonded to each other, at least any one of the plurality of optical elements including a resin lens disposed on a principal surface of a parallel flat glass plate; and
an image pickup member that receives an object image brought into focus by the optical member,
wherein a surface of the resin lens and the principal surface around the resin lens are covered with a transparent inorganic film such that a boundary between the surface of the resin lens and the principal surface around the resin lens is also covered with the transparent inorganic film.

2. The image pickup apparatus for endoscope according to claim 1, wherein at least any one of the resin lenses of the plurality of optical elements has an outer side surface covered with the transparent inorganic film.

3. The image pickup apparatus for endoscope according to claim 2, further comprising a spacer that defines a gap between any two of the optical elements,
wherein the resin lens having the outer side surface covered with the transparent inorganic film is a concave lens, and the surface of the resin lens covered with the transparent inorganic film comprises an optical path surface, an outer circumferential surface that surrounds the optical path surface, and a surface bonded to the spacer.

4. The image pickup apparatus for endoscope according to claim 1, further comprising a spacer that defines a gap between any two of the optical elements,
wherein the principal surface around the resin lens includes a surface bonded to the spacer.

5. An endoscope comprising an image pickup apparatus for endoscope,
wherein the image pickup apparatus for endoscope comprises:
an optical member that is a hybrid lens element in which a plurality of optical elements are bonded to each other, at least any one of the plurality of optical elements including a resin lens disposed on a principal surface of a parallel flat glass plate; and
an image pickup member that receives an object image brought into focus by the optical member, and
wherein a surface of the resin lens and the principal surface around the resin lens are covered with a transparent inorganic film such that a boundary between the surface of the resin lens and the principal surface around the resin lens is also covered with the transparent inorganic film.
